(19) United States
(12) Patent Application Publication
(10) Pub. No.: US 2007/0269829 A1
(43) Pub. Date: Nov. 22, 2007

(54) Instrument and method for nucleic acid isolation

(75) Inventors: Yoshihiro Yamashita, Hitachinaka (JP); Toshinari Sakurai, Hitachinaka (JP)

Correspondence Address: MCDERMOTT WILL & EMERY LLP, 600 13TH STREET, N.W., WASHINGTON, DC, 20005-3096, US

(73) Assignee: HITACHI HIGH-TECHNOLOGIES CORP.

(21) Appl. No.: 11/798,806

(22) Filed: May 17, 2007

(30) Foreign Application Priority Data
May 19, 2006 (JP) 2006-140255

(51) Int. Cl. C12Q 1/68; B01D 15/08; C12M 3/00

(52) U.S. Cl. 435/6; 435/287.2; 210/656

(57) ABSTRACT

A nucleic acid isolation apparatus and method enables nucleic acid isolation from a nucleic-acid-containing sample quickly with high nucleic acid yield and purity. A nucleic acid isolation instrument 11 is supported by a stand 41 via a rib 19 (A). A solution is loaded into a first container 15 via a first opening portion 14 with a loading pipette tip 42. A pressurizing/depressurizing device 43 for controlling the pressure inside the first container 15 and the second container 13 individually is closely fitted in the first opening portion 14 and the second opening portion 12 via a connecting member 44 (B). The pressurizing/depressurizing device 43 pressurizes the inside of the first container 15 while it depressurizes the inside of the second container 13 so as to move the solution from the container 15 to the container 13 via a solid phase 16 (C). After the operation of moving the solution through the solid phase 16 is conducted an appropriate number of times, the pressurizing/depressurizing device 43 is detached from the first and second opening portions 14 and 12. The solution that has moved into the second container 13 is aspirated with an unloading pipette tip 45 via the second opening portion 12 and then unloaded out of the instrument 11 (D).

### BACKGROUND OF THE INVENTION

1. Field of the Invention

The present invention relates to an apparatus and method for isolating nucleic acid from a sample that contains nucleic acid.

2. Background Art

Analysis of nucleic acid, which is a substance that carries genetic information, is being carried out in a wide variety of fields, such as academic research and medicine. Nucleic acid analysis often involves nucleic acid amplification techniques, typically polymerase chain reaction (PCR). PCR is a method of amplifying nucleic acid in a base sequence-specific manner, and it allows detection, quantification, and the like of a target gene.

When performing a nucleic acid analysis such as PCR, it is necessary to isolate nucleic acid from a biological sample or the like and purify it to such an extent that it does not contain substances inhibiting analysis reaction.

In one method of isolating nucleic acid from a biological sample, the property of nucleic acid to become adsorbed on a silica-containing solid phase in the presence of a chaotropic agent is taken advantage of (B. Vogelstein and D. Gillespie, Proc. Natl. Acad. Sci. USA, 76(2), 615-619 (1979)). This property is also utilized by another method whereby nucleic acid is isolated using a nucleic acid isolation instrument having a pipe in which a silica-containing solid phase is placed (JP Patent Publication (Kokai) No. 11-127854 A (1999) and JP Patent Publication (Kokai) No. 2005-241299 A).

In this method, a biological sample and a chaotropic agent are mixed to release nucleic acid from the biological sample. A solution containing the nucleic acid and the chaotropic agent is subjected to an aspiration/discharge operation using the nucleic acid isolation instrument so as to cause the mixture solution to come into contact with the solid phase, thereby allowing the nucleic acid to become adsorbed on the solid phase. Thereafter, a solution to eliminate impurities from the solid phase is subjected to an aspiration/discharge operation using the nucleic acid isolation instrument so as to cause the solution to come into contact with the solid phase to eliminate impurities from the solid phase. Then, a solution to elute the nucleic acid from the solid phase is subjected to an aspiration/discharge operation using the nucleic acid isolation instrument, thereby allowing the solution to come into contact with the solid phase and causing the nucleic acid to be eluted from the solid phase.

### SUMMARY OF THE INVENTION

In the method disclosed in the aforementioned patent publications, the aspiration and discharge of the solution in the nucleic acid isolation instrument allows the solution to come into contact with the solid phase at improved contact efficiency, whereby the efficiency of binding of nucleic acid to solid phase, washing, and elution, can be improved and nucleic acid isolation can be performed with high yield and purity.

However, in the above method, the operation of causing the solution containing a biological sample and having a high viscosity to pass through the solid phase for aspiration and discharge takes a long time. Particularly, in the aspiration operation, since the pressure difference between the inside and outside of the nucleic acid isolation instrument is no more than 1 atmospheric pressure at a maximum, the aspiration force is limited, resulting in an extended period of aspiration time and extending the time required for the entire nucleic acid isolation process.

It is therefore an object of the invention to provide an apparatus and method for nucleic acid isolation whereby nucleic acid can be quickly isolated from a nucleic-acid-containing sample with high nucleic acid yield and purity.

In accordance with the invention, a first container and a second container are communicated with each other via an opening in which a nucleic acid adsorption portion is disposed. One of the first and the second containers is pressurized while the other container is depressurized so as to reciprocate the sample solution through the nucleic acid adsorption portion.

### EFFECTS OF THE INVENTION

In accordance with the invention, nucleic acid can be isolated from a nucleic-acid-containing sample quickly with high nucleic acid yield and purity.

### BRIEF DESCRIPTION OF THE DRAWINGS

FIG. 1 schematically shows a nucleic acid isolation instrument used in a nucleic acid isolation apparatus according to one embodiment of the invention.

FIG. 2 shows a cross section taken along line A-A of FIG. 1.

FIG. 3 shows a cross section taken along line B-B of FIG. 1.

FIG. 4 illustrates an operation for nucleic acid isolation using the nucleic acid isolation instrument.

FIG. 5 shows another example of the nucleic acid isolation instrument of the invention.

FIG. 6 shows yet another example of the nucleic acid isolation instrument of the invention.

FIG. 7 shows yet another example of the nucleic acid isolation instrument of the invention.

FIG. 8 shows a process for nucleic acid isolation according to the invention.

FIG. 9 shows a nucleic acid isolation apparatus according to the invention.

FIG. 10 shows a schematic diagram of a solution loading unit of the nucleic acid isolation apparatus.

FIG. 11 shows a schematic diagram of a pressurizing/depressurizing unit of the nucleic acid isolation apparatus.

FIG. 12 shows a schematic diagram of a solution unloading unit of the nucleic acid isolation apparatus.

FIG. 13 shows a block diagram of the electric system of the nucleic acid isolation apparatus.

FIG. 14 shows a table of the results of a verification experiment comparing the method of the invention with other methods.

### DESCRIPTION OF THE PREFERRED EMBODIMENTS OF THE INVENTION

In the following, embodiments of the invention are described with reference to the drawings.

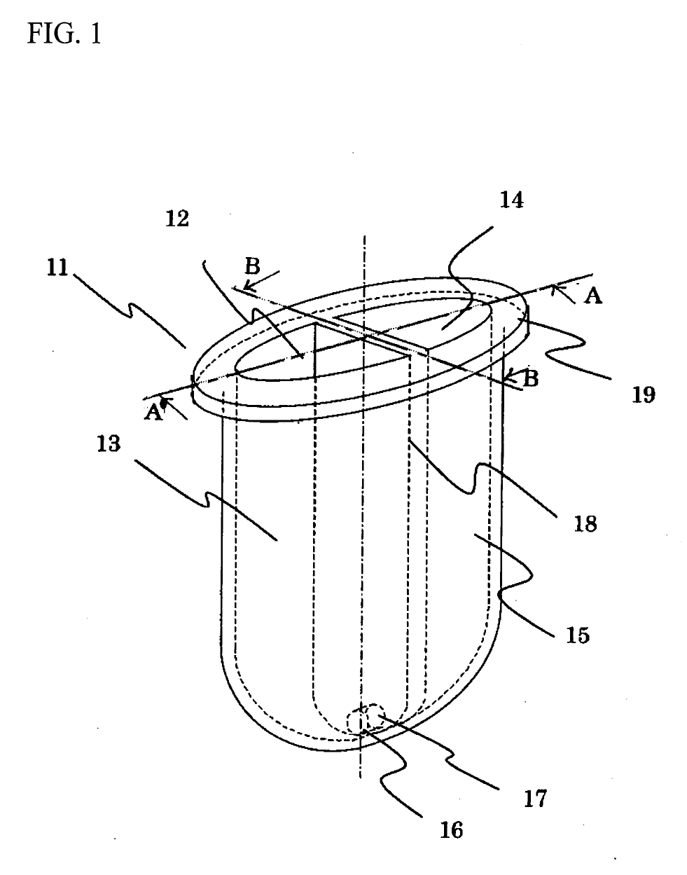

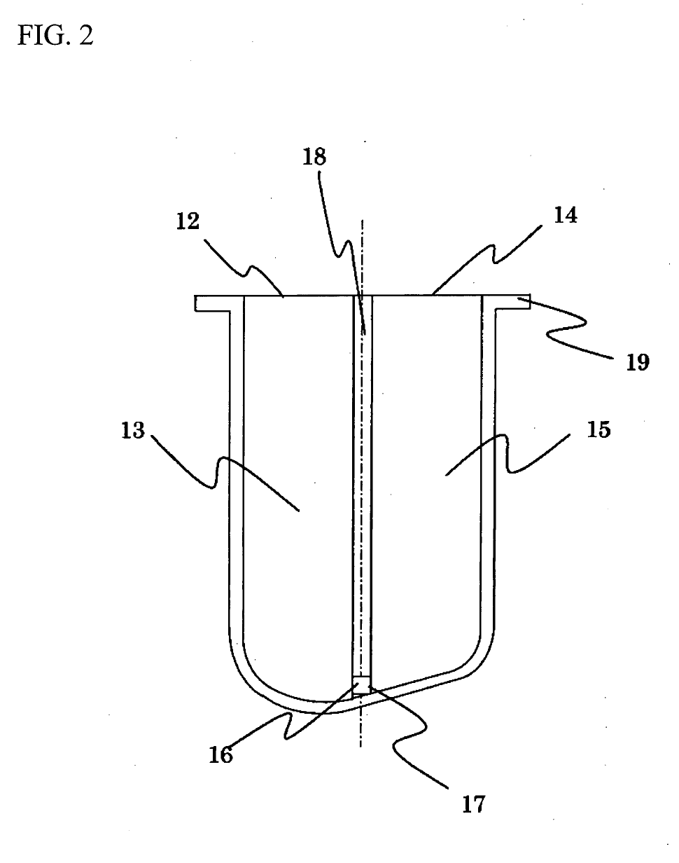

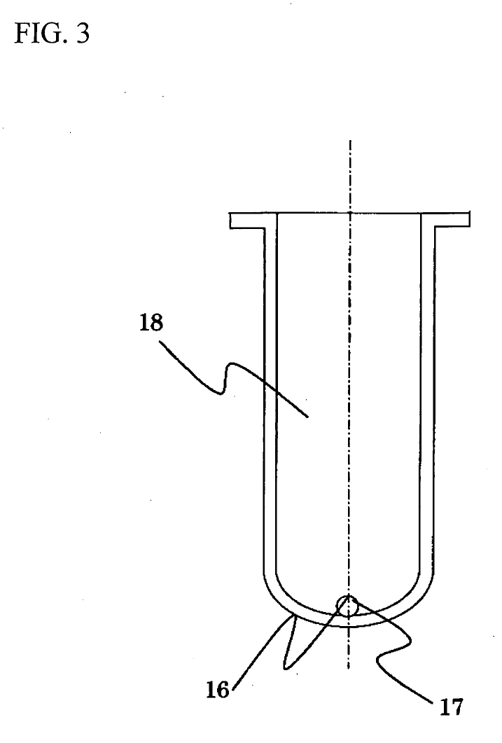

FIG. 1 schematically shows a nucleic acid isolation instrument 11 (nucleic acid isolation container) used in a nucleic acid isolation apparatus according to an embodiment of the invention. FIG. 2 shows a cross section taken along line A-A of FIG. 1; FIG. 3 shows a cross section taken along line B-B of FIG. 1.

Throughout FIGS. 1 to 3, the nucleic acid isolation instrument 11 of the present embodiment consists of two containers 15 and 13 having a first opening portion 14 and a second opening portion 12 at the top, respectively. The two containers are connected at the bottom by a channel 17 in which a solid phase 16 for the adsorption of nucleic acid is inserted.

Thus, the nucleic acid isolation instrument 11 is composed of the first container 15 having the first opening portion 14 and the second container 13 having the second opening portion 12. The first and second containers are isolated by a partition 18 having the connecting channel 17 with the solid phase 6 inserted therein. A disc-shaped rib 19 is provided on the circumference of the openings 12 and 14.

To the first opening portion 14 and the second opening portion 12, a pressurizing/depressurizing device can be detachably attached. When the pressurizing/depressurizing device is attached, the first container 15 and the second container 13 can be hermetically sealed. A pipette tip or the like for dispensing or aspirating a solution can be inserted into the first container 15 or the second container 13 through the first opening portion 14 or the second opening portion 12, respectively.

The solid phase 16 is inserted such that its side faces are closely fitted against the inner walls of the connecting channel 17, so that the solution passing through the connecting channel 17 does so by passing through the inside of the solid phase 16. The rib 19 is a structure for allowing the nucleic acid isolation instrument 11 to be supported by a stand or the like. It also functions to maintain a certain area of contact between the nucleic acid isolation instrument 11 and the pressurizing/depressurizing device when it is attached, to improve their hermetic sealing.

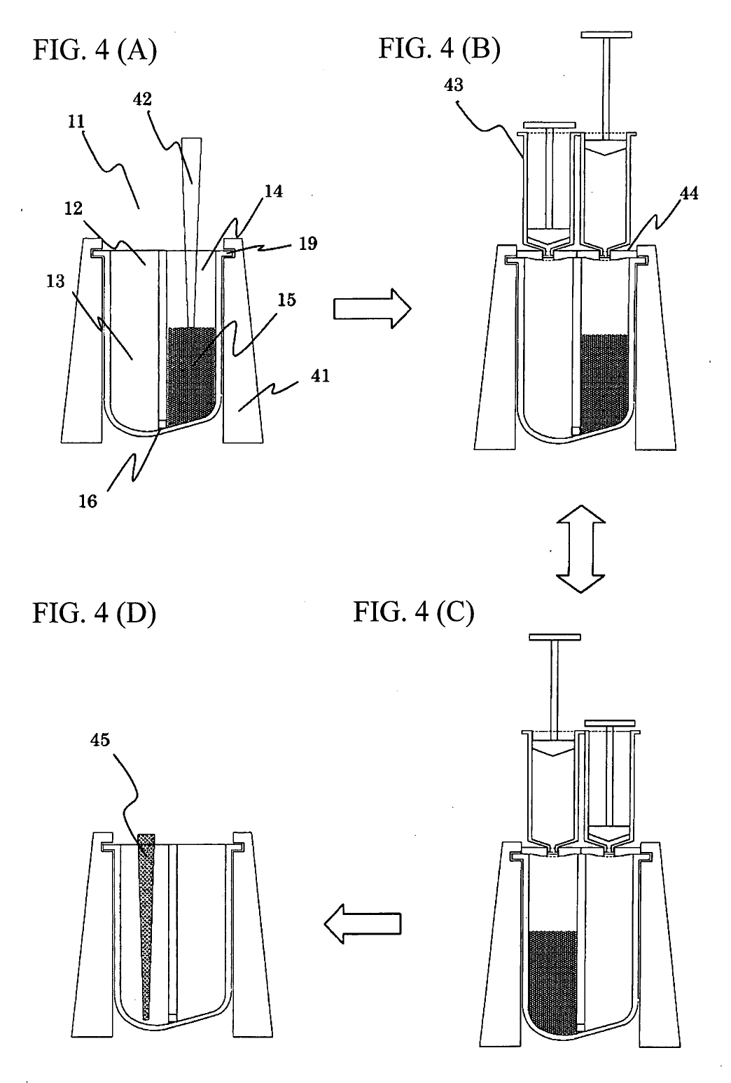

In the following, a method for causing the solution to reciprocate through the solid phase in the nucleic acid isolation instrument 11 of the invention is described with reference to FIG. 4.

FIG. 4(A) shows the nucleic acid isolation instrument 11 being mounted on a stand 41 via the rib 19. A solution is put into the first container 15 with a loading pipette tip 42 through the first opening portion 14.

Then, as shown in FIG. 4(B), a pressurizing/depressurizing device 43 for controlling the pressure inside the first container 15 and the second container 13 individually is closely attached to the first opening portion 14 and the second opening portion 12 via a connecting member 44. The inside of the first container 15 is then pressurized by the pressurizing/depressurizing device 43 via the first opening portion 14, as shown in FIG. 4(C) while the inside of the second container 13 is depressurized by the pressurizing/depressurizing device 43 via the second opening portion 12. As a result, the solution moves from the first container 15 to the second container 13 through the connecting channel 17.

Then, as shown in FIG. 4(B), the inside of the first container 15 is depressurized by the pressurizing/depressurizing device 43 via the first opening portion 14 while the inside of the second container 13 is pressurized by the pressurizing/depressurizing device 43 via the second opening portion 12, whereby the solution moves from the second container 13 to the first container 15 via the connecting channel 17.

After such operation of causing the solution to move from the first container 15 to the second container 13 through the connecting channel 17 has been similarly performed an appropriate number of times, the pressurizing/depressurizing device 43 is detached from the first opening portion 14 and the second opening portion 12. Thereafter, as shown in FIG. 4(D), the solution that has moved to the second container 13 is aspirated by an unloading pipette tip 45 inserted into the second container 13 through the second opening portion 12 and then carried outside the instrument 11.

The opening of the container 15 connecting to the connecting channel 17 is positioned at the bottom-most portion of the container 15, so that the solution moving to the second container 13 can be collected at the connecting channel 17 in a concentrated manner. On the other hand, the opening of the second container 13 connecting to the connecting channel 17 is positioned at the same height as or lower than the corresponding opening of the first container 15, and yet higher than the bottom-most portion of the second container 13.

In this way, the solution inside the first container 15 can move into the second container 13 through the connecting channel 17 efficiently without remaining in the first container 15. The solution that has moved into the second container 13 is aspirated by the pipette tip 45, which is inserted into the second container 13 and which has its tip contacting the bottom-most portion of the second container 13. The solution is then carried outside the instrument efficiently without remaining in the second container 13.

Thus, the solution placed in the second container 13 or the first container 15 is eventually entirely moved to the second container 13, from which the entire amount of it is further aspirated by the pipette tip 45 inserted into the second container 13 through the second opening portion 12 and carried outside the instrument 11.

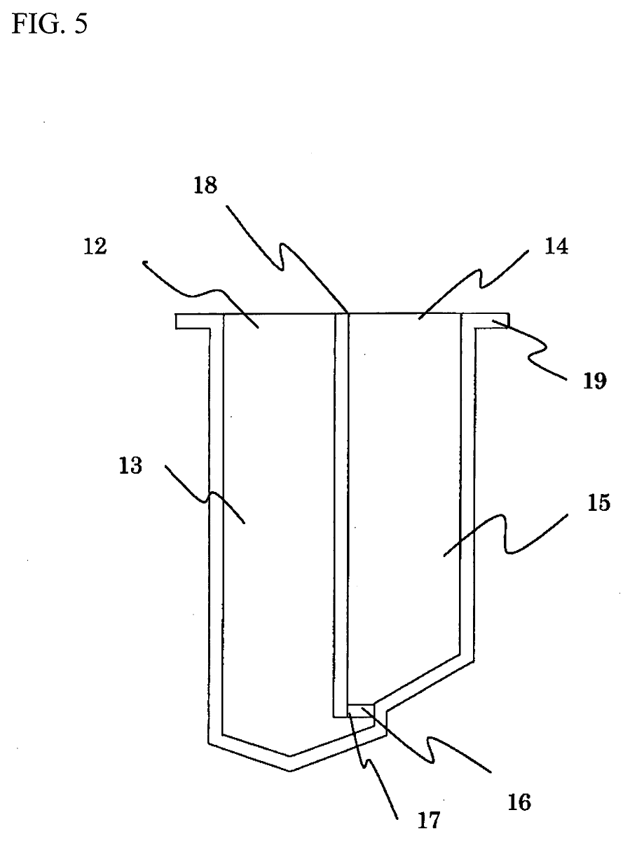

The nucleic acid isolation instrument 11 as described above may be configured as shown in FIGS. 5 to 7, in addition to the configuration of FIG. 1.

FIG. 5 shows an example in which the solid phase 16 is fixed at the bottom-most portion of the first container 15, and the central axis of the connecting channel 17 extends along the central axis of a cylindrical or rectangular-cylindrical container 15, so that the solution can move in the connecting channel 17 along the central axis.

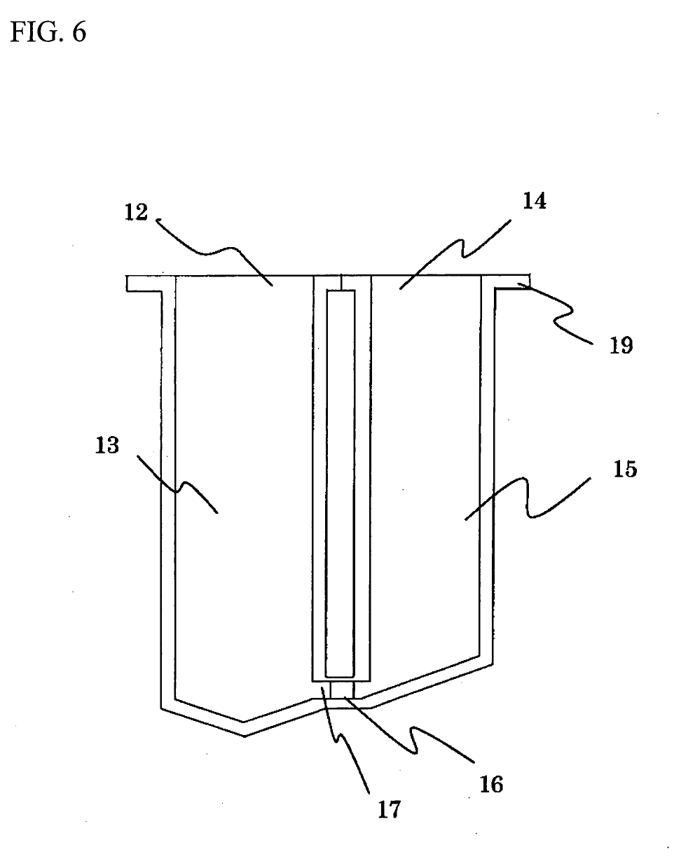

FIG. 6 shows another example in which the first container 15 and the second container 13 do not share a partition but constitute independent containers, which are connected by a coupled by a channel 17. In this example, the central axis of the connecting channel 17 extends in a direction substantially perpendicular to the central axis of the cylindrical or rectangular-cylindrical container 15, as in the example of FIG. 1.

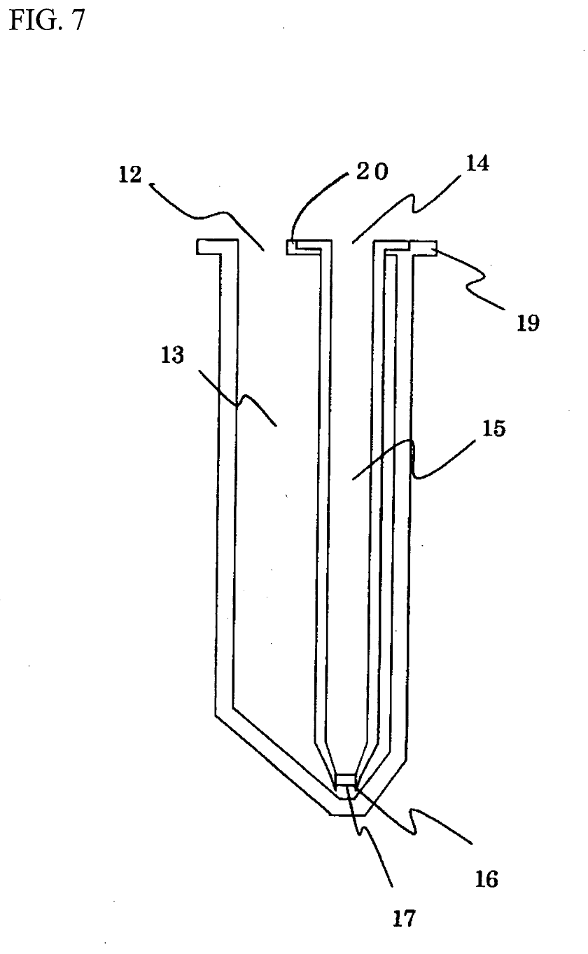

Another example is shown in FIG. 7 consisting of a tubular first container 15 and the second container 13, in which the solid phase 16 is fixed at the tip (on the bottom) of the first container 15, the first container 15 being contained within the second container 13.

In this example, the first container 15 and the second container 13 can be attached to and detached from each other via a connecting portion 20. The first container 15 with a solution in it can be detached from the second container 13 while depressurized by the pressurizing/depressurizing device connected to the first container 15, and the thus detached first container 15 can be moved to a predetermined position. By then pressurizing the inside of the first container 15 with the pressurizing/depressurizing device, the solution can be moved from the first container 15 to a predetermined position. Thus, in this example, the first container 15 also functions as a pipette tip or the like to carry the solution outside the container.

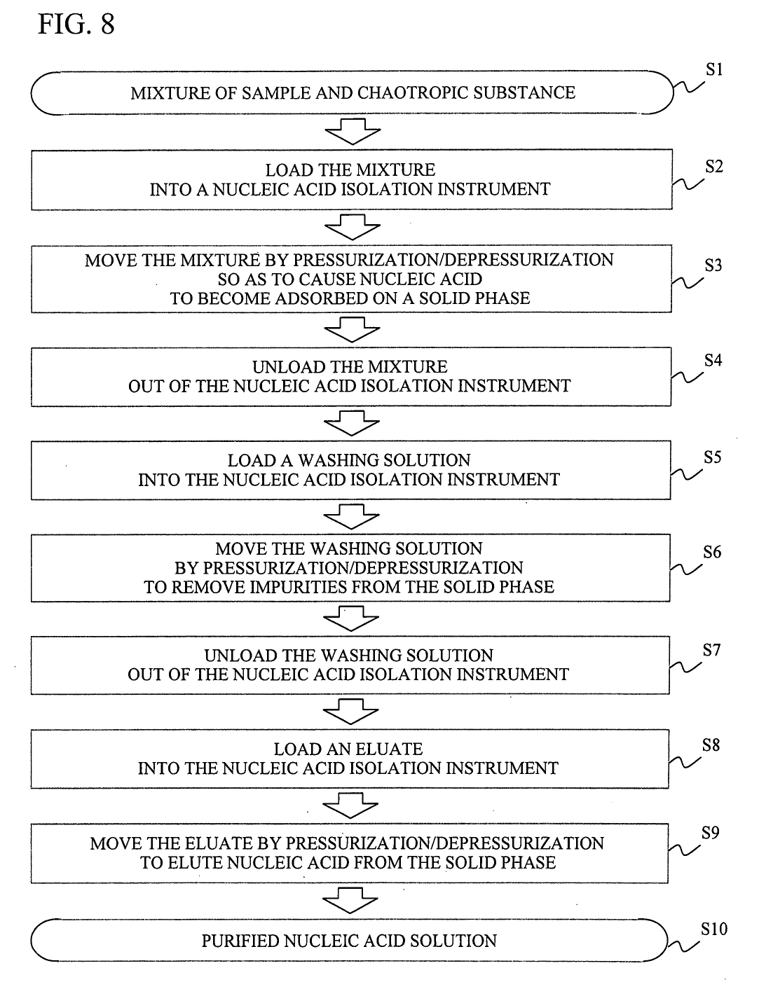

In the following, a process of nucleic acid isolation using the nucleic acid isolation instrument 11 of the invention is described with reference to FIG. 8.

In step S1 of FIG. 8, a nucleic-acid-containing sample, a chaotropic agent, and, as needed, a substance to promote the release of nucleic acid from the sample and a substance to promote the adsorption of nucleic acid on the silica-containing solid phase, are mixed.

After the mixture solution is placed in the first container 15 of the nucleic acid isolation instrument 11, the pressurizing/depressurizing device is connected to move the mixture solution so as to cause the nucleic acid to become adsorbed on the solid phase. The pressurizing/depressurizing device is then detached, and the mixture solution that has moved to the second container 13 is carried outside the nucleic acid isolation instrument 11 (steps S2 to S4).

Thereafter, a washing solution to remove impurities in the solid phase 16 is put into the first container 15 of the nucleic acid isolation instrument 1, and the pressurizing/depressurizing device is connected to move the mixture solution and remove impurities in the solid phase 16. The pressurization/depressurization device is then detached, and the washing solution that has moved into the second container 13 is carried outside the nucleic acid isolation instrument (steps S5 to 7).

A solution to elute nucleic acid from the solid phase 16 is then put into the first container 15 of the nucleic acid isolation instrument 11, and the pressurizing/depressurizing device is connected to move the mixture solution so as to elute nucleic acid from the solid phase 16. The pressurization/depressurization device is then detached, and the solution that has moved into the second container 13 is collected as purified nucleic acid solution (steps S8 to 10).

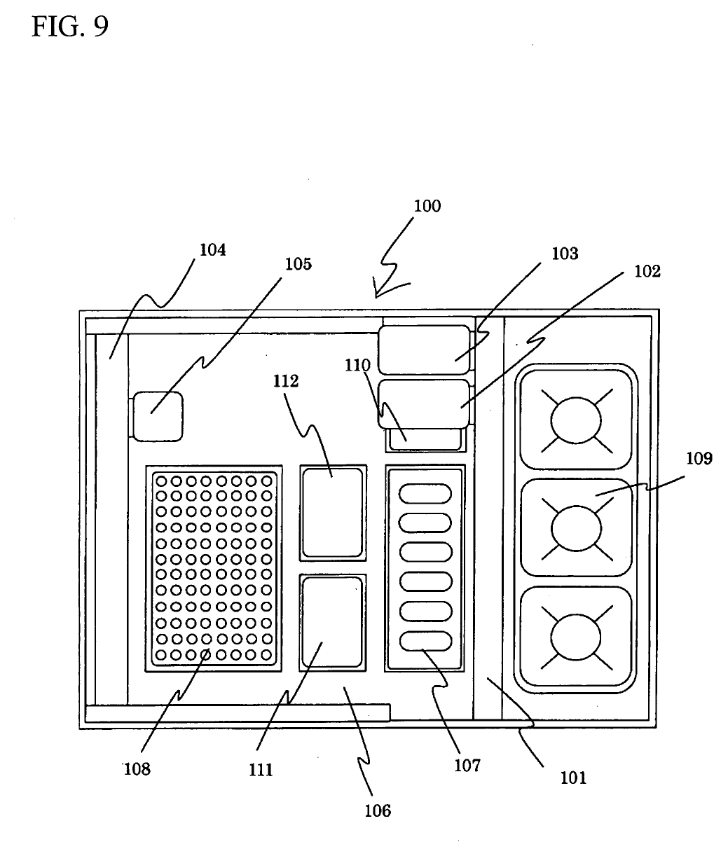

In the following, the overall configuration of a nucleic acid isolation apparatus employing the nucleic acid isolation instrument of the invention is described with reference to FIGS. 9 to 13.

FIG. 9 is a schematic plan view of the nucleic acid isolation apparatus of the invention. As shown, the nucleic acid isolation apparatus 100 includes a solution loading unit 102 and a pressurizing/depressurizing unit 103 that are mounted on a rail 101. The solution loading unit 102 is movable along a Y-axis on the rail 101. The pressurizing/depressurizing unit 103 is movable along both the Y-axis and a Z-axis.

The apparatus also includes an arm 104 that is movable along an X-axis. On the arm 104, a solution unloading unit 105 is mounted movably along the Y-axis, which lies along the arm 104, and along the Z-axis. On a working surface 106 of the main body rack, there are disposed a stand 107 for supporting the nucleic acid isolation instrument 11; a rack 108 for unused pipette tips; reagent bottles 109; a reagent receiver 110 for reagent priming; a liquid waste receiver 111 for mixture solution and washing solution; and a used pipette tip receiver 112.

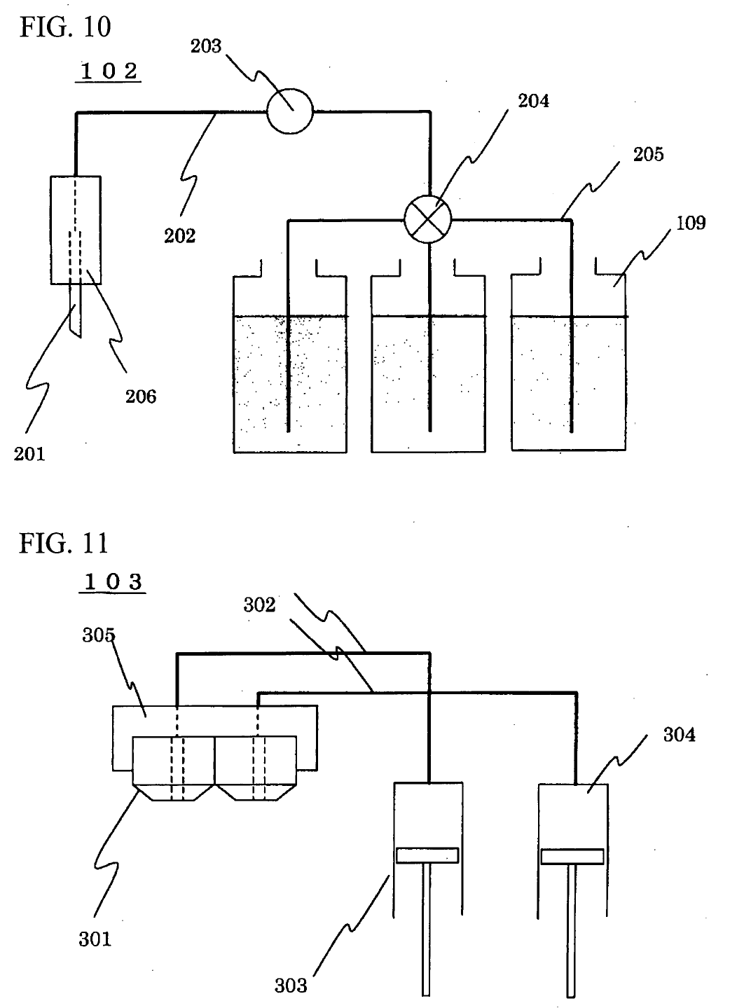

FIG. 10 shows a schematic diagram of the solution loading unit 102. As shown, the solution loading unit 102 includes: a nozzle 201 for discharging the solution; a nozzle holder 206 for holding the nozzle 201; a pump 203 for aspirating the reagent; a switching valve 204 for switching the reagent; a pipe 202 communicating the switching valve 204 and the nozzle 201; and a pipe 205 for communicating the switching valve 204 and each reagent.

The solution loading unit 102 moves on the rail 101 along the Y-axis and is positioned above the reagent receiver, where it activates the switching valve 204 and selects a predetermined reagent, of which a predetermined amount is primed. The solution loading unit 102 then moves along the axis and is positioned above a predetermined nucleic acid isolation instrument 11 supported by the stand 107, where it discharges a predetermined amount of reagent via the nozzle 201, thereby loading the nucleic acid isolation instrument 11 with the reagent.

FIG. 11 shows a schematic diagram of the pressurizing/depressurizing unit 103. As shown, the pressurization/depressurization unit 103 includes: a connecting member 301 for connecting syringes 303 and 304, which constitute the pressurizing/depressurizing device 43, and the openings 12 and 14 of the nucleic acid isolation instrument 11; pipes 302 communicating the syringes 303 and 304 with the connecting member 301; and a connecting portion holder 305 for holding the connecting member 301.

The pressurizing/depressurizing unit 103 moves on the rail 101 along the Y-axis and is positioned above a predetermined nucleic acid isolation instrument 11 carried in the stand 107. The unit then moves downward along the Z-axis, has its connecting member 301 closely fitted against the nucleic acid isolation instrument 11, and pressurizes or depressurizes the inside of each of the containers 13 and 15 at a predetermined pressure by means of the syringes 303 and 304. After the solution has moved, the unit moves upward along the Z-axis so as to detach the connecting member 301 from the nucleic acid isolation instrument 11.

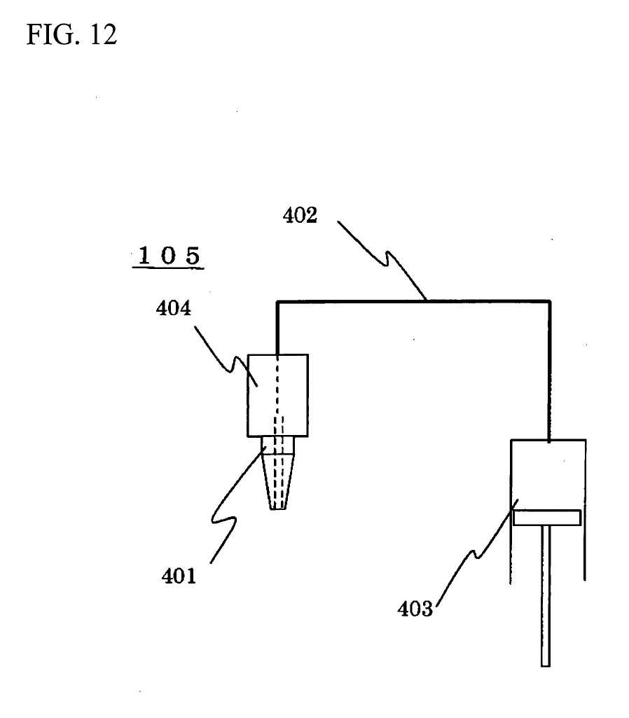

FIG. 12 shows a schematic diagram of the solution unloading unit 105. As shown, the solution unloading unit 105 includes: a connecting portion 401 for connection with a tip 45; a connecting portion holder 404 for holding the connecting portion 401; a syringe 403 for the aspiration/discharge of a solution; and a pipe 402 communicating the connecting portion 401 and the syringe 403.

The solution unloading unit 105 moves along the X- and Y-axes to a predetermined position above the pipette tip rack 108, where it then moves downward along the Z-axis to have a pipette tip pressed into the connecting portion 401. The unit then raises the pipette tip along the Z-axis, and moves along the X- and Y-axes and is positioned above a predetermined nucleic acid isolation instrument 11 supported by the stand 107, where the unit lowers itself along the Z-axis to insert the pipette tip into the nucleic acid isolation instrument 11. Then, the unit aspirates the solution inside the nucleic acid isolation instrument 11, raises the pipette tip upward along the Z-axis, and moves along the X- and Y-axes. After positioned above the liquid waste receiver 111, the solution unloading unit 105 lowers the pipette tip downward along the Z-axis to insert it into the liquid waste receiver 111, where the solution is discharged.

The solution unloading unit 105 then raises the pipette tip along the Z-axis, moves along the X- and Y-axes until it is positioned above the used pipette tip receiver 112, where it lowers the pipette tip along the Z-axis to insert it into the used pipette tip receiver 112. The unit then moves along the X-axis so as to insert the connecting portion 401 into a U-shaped groove provided within the pipette tip receiver 112. The unit then moves upward along the Z-axis, whereby the pipette tip is removed from the connecting portion 401 as the upper opening of the pipette tip is caught in the U-shaped groove, thereby discarding the pipette tip in the pipette tip receiver 112.

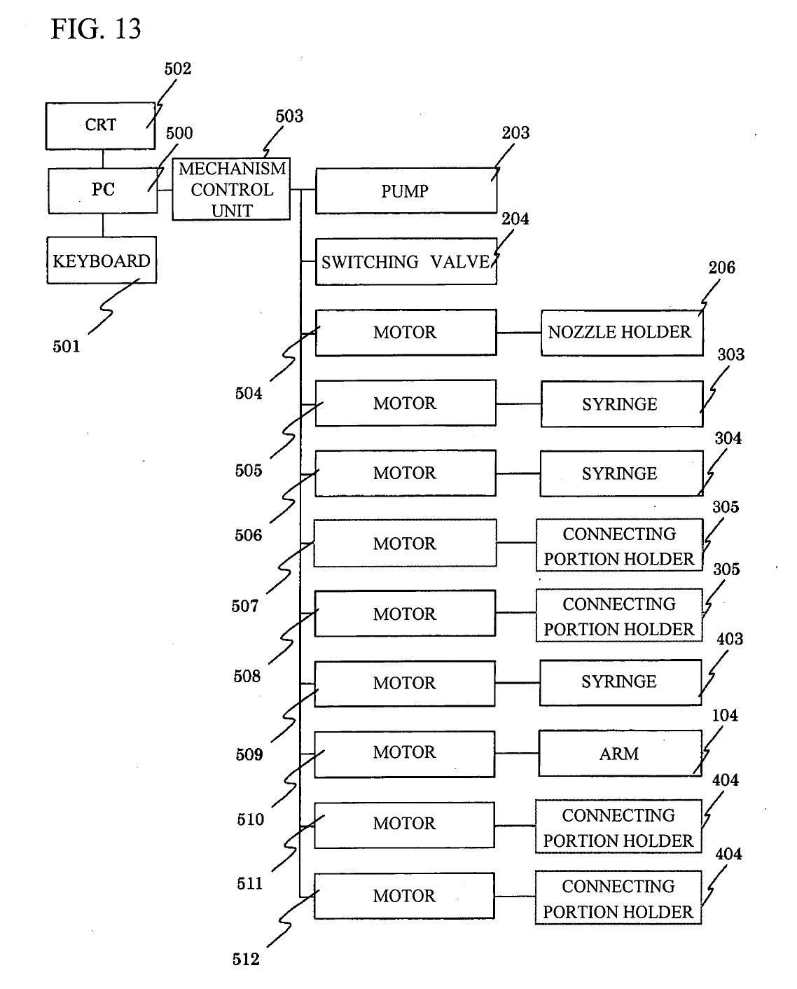

FIG. 13 shows a schematic block diagram of the electric system of the nucleic acid isolation apparatus of the invention. As shown, a personal computer (PC) 500, as an operation control unit, has connected thereto: an operating panel and keyboard 501 for the entry of operating conditions or the like; a display unit (CRT) 502 for displaying input information and operating status; and a mechanism control unit 503 for controlling the individual mechanism units of the apparatus.

The mechanism control unit 503 controls: the pump 203 for discharging solution; the switching valve 204 for switching the type of reagent; the motor 504 for moving the nozzle holder 206 along the Y-axis; and motors 505 and 506 for driving the pistons for carrying out pressurization/depressurization by means of the syringes 303 and 304. Further, the mechanism control unit 503 controls: a motor 507 for moving the connecting portion holder 305 along the Y-axis; a motor 508 for moving the connecting portion holder 305 along the Z-axis; a piston-driving motor 509 for the aspiration/discharge of solution by means of the syringe 403; a motor 510 for moving the arm 104 carrying the solution unloading unit 105 along the X-axis; a motor 511 for moving the connecting portion holder 404 along the Y-axis; and a motor 512 for moving the connecting portion holder 404 along the Z-axis.

Each portion of the nucleic acid isolation apparatus is operated in accordance with an instruction from the PC based on a predetermined program.

The nucleic-acid-containing sample may consist of whole blood, blood serum, blood plasma, sputum, urine, saliva, semen or other tissues, or body fluid containing a cell, bacteria, virus, or the like. It may also consist of a cultured cell, cultured bacteria, coarsely purified nucleic acid, or the like.

The chaotropic agent may consist of sodium iodide, potassium iodide, sodium perchlorate, sodium thiocyanate, guanidine thiocyanate, or guanidine hydrochloride, for example. The chaotropic agent is a substance for promoting the release of nucleic acid from the sample and the adsorption of nucleic acid on a solid phase.

The release of nucleic acid from the sample can also be promoted by: a physical method involving ultrasound, agitation or the like; a chemical method involving a surface active agent, protein denaturant or the like; a biochemical method involving protein degrading enzyme or the like; or a combination thereof.

The adsorption of the nucleic acid on the silica-containing solid phase can also be promoted by an organic solvent mixture. The organic solvent may consist of one or more compounds selected from the group consisting of aliphatic alcohol, aliphatic ether, aliphatic ester, and aliphatic ketone. Examples of aliphatic alcohol that can be used include methanol, ethanol, 2-propanol, 2-butanol, and polyethylene glycol. Examples of aliphatic ether that can be used include diethylene glycol dimethyl ether, diethylene glycol diethyl ether, ethylene glycol dimethyl ether, ethylene glycol diethyl ether, propylene glycol dimethyl ether, propylene glycol diethyl ether, and tetrahydrofuran.

Examples of aliphatic ester that can be used include ethyl lactate and propylene glycol monomethyl ether acetate. Examples of aliphatic ketone that can be used include acetone, hydroxy acetone, and methyl ketone.

The solid phase may consist of a substance that contains silica, such as glass particles, silica particles, glass fiber, silica fiber, and monolithic silica; alternatively, it may consist of an organic polymer with a hydrophilic group, such as a hydroxyl group, on its surface that is formed to have a plurality of continuous pores therein. A connecting member having a sealing property may be disposed between the side faces of the solid phase and the inner walls of the connecting channel so as to allow the solid phase to be closely attached to the inner walls of the connecting channel when it is inserted and to cause the solution to pass through the inside of the solid phase when it passes through the connecting channel.

In order to prevent the solid phase from flowing out of the connecting channel due to the resistance provided by the solution as it moves, a holding member having a fluid-communicating property may be disposed at each end of the solid phase inserted in the connecting channel.

The pressurizing/depressurizing device may consist of a syringe, a pump, or the like for each container, with a volume equal to or greater than the volume of the solution to be moved. Preferably, the pressurizing/depressurizing device and the nucleic acid isolation instrument are connected via a connecting member having a sealing property, so as to maintain the space between the pressurizing/depressurizing device and the nucleic acid isolation instrument hermetically sealed.

A washing solution that can remove impurities that have become bound to the silica-containing solid phase while maintaining the adsorption of the nucleic acid on the silica-containing solid phase is selected. For instance, the washing solution may consist of an ethanol or diethylene glycol dimethyl ether to which a chaotropic agent, a surface active agent, a buffer, or the like have been added; alternatively, it may consist of an aqueous solution of ethanol or diethylene glycol dimethyl ether.

The eluate for eluting the nucleic acid that has become adsorbed on the solid phase must be capable of eluting nucleic acid form the silica-containing solid phase; it may consist, for example, of nuclease-free water or a nuclease-free buffer solution of low salt concentration. The eluate may contain an analysis reaction reagent to be used for a nucleic acid analysis reaction, which will be described later. For example, when PCR is conducted as such nucleic acid analysis reaction, an eluate that contains a PCR reagent (buffer solution, primer, enzyme, or the like) may be used; in this case, a PCR reaction can be initiated immediately from the eluate.

In the following, a verification experiment conducted to examine the effects of the invention is described.

The sample, reagent, and instrument used in the verification experiment are described below.

### 1. Sample

Healthy human blood (Anticoagulant EDTA-2Na added)

### 2. Reagent

### 2.1 Chaotropic Solution

5.5M guanidinium hydrochloride, 80 mM MES, 47 mM EDTA-2Na, 20% (v/v) polyoxyethylenesorbitan monolaurate, and 1% (v/v) Disfoarn

### 2.2 Enzyme Solution

20 mg/ml proteinase K, and 10 mM Tris-HCl (pH 7.5)

### 2.3 Organic Solvent

Diethylene glycol dimethyl ether

### 2.4 Washing Solution A

950 mM guanidinium hydrochloride, 14 mM MES, 8 mM EDTA-2Na, 4% (v/v) polyoxyethylenesorbitan monolaurate, and 30% (v/v) diethylene glycol dimethylether

### 2.5 Washing Solution B

55% (v/v) ethanol, 14 mM acetic acid potassium, and 11 mM acetic acid

### 2.6 Eluate

10 mM Tris-HCl (pH 8.0), and 0.1 mM EDTA (pH 8.0)

### 3. Nucleic Acid Isolation Instrument

### 3.1 Solid Phase

As a solid phase, monolithic silica (diameter 4 mm, length 2 mm, pore size 15 μm) was used.

### 3.2 Nucleic Acid Isolation Instrument of the Invention

A nucleic acid isolation instrument of the invention with the shape shown in FIG. 1 was fabricated from polypropylene resin, each container having a volume of about 1 ml and the connecting channel measuring 4 mm in diameter and 2 mm in length and having the solid phase force-fitted and fixed therein.

### 3.3 A Nucleic Acid Isolation Instrument (Tip-Type Nucleic Acid Isolation Instrument) for a Method Different From the Present Invention

A nucleic acid isolation instrument for a method different from the method of the invention was fabricated from polypropylene resin, the container having a volume of about 1 ml of solution and having a diameter of 4 mm at its tip where the solid phase was force-fitted and fixed. The pressurization/depressurization device for aspirating and discharging the solution was connected to the opening of the tip-type nucleic acid isolation instrument so as to cause the solution to pass through the solid phase.

### 3.4 Pressurizing/Depressurizing Device

As the pressurizing/depressurizing device, a syringe (volume 2.5 ml) was used. The connection of the nucleic acid isolation instrument and the syringe was made by a silicon-resin connecting member adapted to the shape of each connecting portion. For the nucleic acid isolation instrument of the invention, two such syringes and two such connecting members were used; for the tip-type nucleic acid isolation instrument, one such syringe and one such connecting member were used.

The nucleic acid isolation method for the nucleic acid isolation instrument of the invention is as follows:
(1) To 0.2 ml of the whole blood, 0.02 ml of the enzyme solution and 0.24 ml of the chaotropic solution are added and mixed.
(2) Incubate at 50° C. for 20 minutes.
(3) 0.2 ml of the organic solvent is added and mixed.
(4) The mixture solution is put into the nucleic acid isolation instrument.
(5) The pressurizing/depressurizing device is connected to the nucleic acid isolation instrument, and the mixture solution is caused to move back and forth between the individual containers 4.5 times.
(6) The mixture solution is put out of the nucleic acid isolation instrument.
(7) 1.0 ml of the washing solution A is put into the nucleic acid isolation instrument.
(8) The pressurizing/depressurizing device is connected to the nucleic acid isolation instrument so as to cause the mixture solution to move into the other container.
(9) The washing solution is put out of the nucleic acid isolation instrument.
(10) 1.0 ml of the washing solution A is put into the nucleic acid isolation instrument.
(11) The pressurizing/depressurizing device is connected to the nucleic acid isolation instrument so as to move the mixture solution into the other container.
(12) The washing solution is put out of the nucleic acid isolation instrument.
(13) 1.0 ml of the washing solution B is put into the nucleic acid isolation instrument.
(14) The pressurizing/depressurizing device is connected to the nucleic acid isolation instrument so as to move the mixture solution into the other container.
(15) The washing solution is put out of the nucleic acid isolation instrument.
(16) 1.0 ml of the washing solution B is put into the nucleic acid isolation instrument.
(17) The pressurizing/depressurizing device is connected to the nucleic acid isolation instrument so as to move the mixture solution into the other container.
(18) The washing solution is put out of the nucleic acid isolation instrument.
(19) 0.2 ml of the eluate is put into the nucleic acid isolation instrument.
(20) The pressurizing/depressurizing device is connected to the nucleic acid isolation instrument, and the eluate is caused to move back and forth between the containers four to five times.
(21) The eluate is put out of the nucleic acid isolation instrument and collected as a purified nucleic acid solution.

In the following, a nucleic acid isolation method 1 (involving a reciprocating communication of fluid by aspiration and discharge) different from the method of the invention for nucleic acid isolation in a comparative example is described.
(1) To 0.2 ml of the whole blood, 0.02 ml of the enzyme solution and 0.24 ml of the chaotropic solution are added and mixed.
(2) Incubate at 50° C. for 20 minutes.
(3) 0.2 ml of the organic solvent is added and mixed.
(4) The mixture solution is put into the tip-type nucleic acid isolation instrument via its upper opening; the pressurizing/depressurizing device is connected and the mixture solution in the instrument is discharged; and the solution is then aspirated and discharged four times (for a total of 4.5 reciprocations of aspiration and discharge)
(5) The mixture solution is discarded.
(6) 1.0 ml of the washing solution A is put into the instrument via its upper opening and then discharged.
(7) The washing solution is discarded.
(8) 1.0 ml of the washing solution A is put into the instrument via its upper opening.
(9) The washing solution is discarded.
(10) 1.0 ml of the washing solution B is put into the instrument via its upper opening.
(11) The washing solution is discarded.
(12) 1.0 ml of the washing solution B is put into the instrument via its upper opening and then discharged.
(13) The washing solution is discarded.
(14) 0.2 ml of the eluate is put into the instrument via its upper opening; the pressurizing/depressurizing device is connected and the eluate inside the instrument is discharged; the eluate is then aspirated and discharged four times (for a total of 4.5 times of reciprocations of aspiration and discharge)
(15) The eluate is collected as a purified nucleic acid solution.

In the following, a nucleic acid isolation method 2 (involving a one-way communication of fluid by discharge) different from the method of the invention for nucleic acid isolation in a comparative example is described.
(1) To 0.2 ml of the whole blood, 0.02 ml of the enzyme solution and 0.24 ml of the chaotropic solution are added and mixed.
(2) Incubate at 50° C. for 20 minutes.
(3) 0.2 ml of the organic solvent is added and mixed.
(4) The mixture solution is put into the tip-type nucleic acid isolation instrument via its opening; the pressurizing/depressurizing device is connected; and the mixture solution in the instrument is discharged (one-way discharge).
(5) The mixture solution is discarded.
(6) 1.0 ml of the washing solution A is put into the instrument via its opening and then discharged.
(7) The washing solution is discarded.
(8) 1.0 ml of the washing solution A is put into the instrument via its opening and then discharged.
(9) The washing solution is discarded.
(10) 1.0 ml of the washing solution B is put into the instrument via its opening and then discharged.
(11) The washing solution is discarded.
(12) 1.0 ml of the washing solution B is put into the instrument via its opening and then discharged.
(13) The washing solution is discarded.
(14) 0.2 ml of the eluate is put into the instrument via its opening; the pressurizing/depressurizing device is connected; and the eluate in the instrument is discharged (one-way discharge).
(15) The eluate is collected as a purified nucleic acid solution.

In the following, a method of evaluating the nucleic acid purified by the verification experiment is described.

### 1. Determination of the Concentration and Purity of the Nucleic Acid

The concentration of the nucleic acid was determined by measuring its absorbance at 260 nm using a spectrophotometer and then calculating its ratio to the unit absorbance (1.0) of a 50-μg/ml DNA solution at 260 nm. The purity of the nucleic acid was determined by measuring its absorbance at 260 nm and 280 nm using the spectrophotometer and calculating their ratio (A260/A280).

### 2. Measurement of the Time Required for Nucleic Acid Isolation

The time it took for obtaining a nucleic acid solution was measured from a measurement start point, which was a point in time when, after adding the enzyme solution and the chaotropic solution to the sample and incubating it, a mixture solution was prepared by adding the organic solvent.

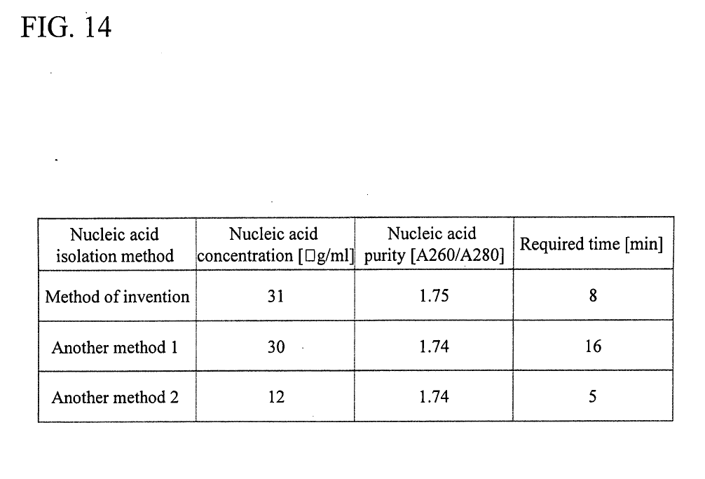

FIG. 14 shows the experimental results comparing the nucleic acid isolation method involving the nucleic acid isolation instrument of the invention and the nucleic acid isolation methods 1 and 2 involving the nucleic acid isolation instrument adapted for such methods, which are different from the method of the invention.

As compared with another method 1, the method of the invention provides equal or higher concentration and purity of nucleic acid. The time required for nucleic acid isolation was 8 minutes for the present invention while 16 minutes for another method 1, thus showing a 50% decrease. Such difference in the required time is mainly due to the duration of time that the mixture solution, which has a large fluid communication resistance, is reciprocated through the solid phase.

Specifically, since the method of the invention causes a pressure difference to be produced by applying a positive pressure and a negative pressure simultaneously at the ends of the solution when reciprocating it through the solid phase, the rate of movement of the solution can be increased. In another method 1, on the other hand, either a positive pressure or a negative pressure alone is produced at one end of the solution when reciprocating it through the solid phase, with the result that the pressure difference produced is small. Particularly, if only a negative pressure alone is applied, the pressure difference is less than one atmospheric pressure at most, such that the aspiration force is limited and the rate of movement of the solution decreases.

As compared with another method 2, the method of the invention provides 2.6-times higher concentration and an equal or better purity of nucleic acid. The time required for nucleic acid isolation, however, was 1.6 times longer for the method of the invention than another method 2, which required five minutes. In accordance with the method of the invention, the efficiency of binding of nucleic acid and solid phase and the efficiency of elution are improved by causing the mixture solution, which contains the sample and the chaotropic substance, and the eluate to reciprocate through the solid phase, thereby improving the nucleic acid yield.

On the other hand, in another method 2, the solution is caused to pass through the solid phase in only one direction, so that, although the required time can be reduced, the binding efficiency and the elution efficiency decrease, resulting in a decrease in nucleic acid yield.

Furthermore, in accordance with the method of the invention, since the reciprocating movement of the solution occurs within the sealed container separated from the pressurizing/depressurizing device, no droplets or the like of the solution are allowed to escape to the outside as the solution is moved, thereby reducing the possibility of polluting the environment.

On the other hand, in methods 1 and 2, droplets of the solution are produced as the solution is discharged from within the nucleic acid isolation instrument to the outside, thus increasing the possibility of environmental pollution.

Thus, in accordance with one embodiment of the invention, the nucleic acid isolation instrument 11 includes the two container portions 13 and 15 each having an opening. The containers 13 and 15 are communicated at the bottom thereof by the connecting channel 17, in which the solid phase 16 for the adsorption of nucleic acid is disposed. The sample solution is put in one of the containers 13 and 15, and one container is pressurized while the other container is depressurized repeatedly by means of the pressurizing/depressurizing device 43, thereby allowing the sample solution to reciprocate through the solid phase within the sealed containers quickly.

Thus, the invention provides a apparatus, method, and an instrument for nucleic acid isolation whereby nucleic acid can be isolated from a nucleic-acid-containing sample quickly with high nucleic acid yield and purity within a sealed instrument.

What is claimed is:

1. An apparatus for isolating nucleic acid from a sample solution, comprising:
a solid phase for the adsorption of nucleic acid in the sample solution;
a nucleic acid isolation instrument comprising a first container portion and a second container portion that are communicated with each other via the solid phase wherein a solution is loaded into and unloaded out of the nucleic acid isolation instrument; and,
pressurization/depressurization device for pressurizing the inside of one of the first container portion and the second container portion of the nucleic acid isolation instrument while depressurizing the inside of the other container portion.

2. The nucleic acid isolation apparatus according to claim 1, wherein the pressurization/depressurization device comprises a first syringe pump connected to the first container portion and a second syringe pump connected to the second container portion.

3. The nucleic acid isolation apparatus according to claim 1, wherein the sample solution is loaded into the nucleic acid isolation instrument and is unloaded out of the nucleic acid isolation instrument after a pressurization/depressurization operation by the pressurization/depressurization device, wherein a washing solution is loaded into the nucleic acid isolation instrument and the washing solution is unloaded out of the nucleic acid isolation instrument after a pressurization/depressurization operation by the pressurization/depressurization device and wherein an eluate is loaded into the nucleic acid isolation instrument and the eluate is unloaded out of the nucleic acid isolation instrument after a pressurization/depressurization operation by the pressurization/depressurization device.

4. A method of isolating nucleic acid from a sample solution, comprising:
loading a sample solution into either a first container portion or a second container portion that are communicated via a solid phase for the adsorption of nucleic acid in the sample solution;
pressurizing the inside of one of the first and the second container portions while depressurizing the other container portion so as to cause the sample solution to be reciprocated between the first container portion and the second container portion through the adsorption means and to thereby allow nucleic acid in the sample solution to become adsorbed on the solid phase; and
isolating the nucleic acid that has become adsorbed on the solid phase.

5. The nucleic acid isolation method according to claim 4, comprising:
loading, after the nucleic acid has become adsorbed on the solid phase, a washing solution into one of the first and the second container portions;
carrying out a pressurization/depressurization operation by pressurizing the inside of one of the first and the second container portions while depressurizing the inside of the other container portion so as to cause the washing solution to be moved between the first container portion and the second container portion through the solid phase;
unloading the washing solution out of a nucleic acid isolation instrument having the first container portion and the second container portion;
loading an eluate into one of the first and the second container portions;
unloading, after a pressurization/depressurization operation, the eluate out of the nucleic acid isolation instrument having the first container portion and the second container portion; and
isolating the nucleic acid.

6. An instrument for isolating nucleic acid from a sample solution, comprising:
a container;
a partition dividing the container into a first container portion having a first opening portion and a second container having a second opening portion, the partition comprising a communication opening formed therein, the communication opening communicating the first and the second container portions with each other; and
a solid phase for the adsorption of nucleic acid, the solid phase being disposed within the communication opening formed in the partition.

7. The nucleic acid isolation instrument according to claim 6, wherein the communication opening is formed at a position corresponding to the bottom of the first container portion and the second container portion.

8. The nucleic acid isolation instrument according to claim 6, wherein the bottom of the first container portion is inclined toward the bottom of the second container portion.

9. The nucleic acid isolation instrument according to claim 8, comprising a disc-shaped rib formed at the opening portion of the first container portion and the second container portion.

10. An instrument for isolating nucleic acid from a sample solution, comprising:
a container;
a partition dividing the container into a first container portion having a first opening portion and a second container portion having a second opening portion; and
a solid phase for the adsorption of nucleic acid,
wherein the first container portion comprises a communication opening formed at its bottom, the communication opening communicating the first container portion and the second container portion with each other, and wherein the solid phase is disposed within the communication opening.

11. The nucleic acid isolation instrument according to claim 10, comprising a disc-shaped rib formed at the opening portions of the first container portion and the second container portion.

12. An instrument for isolating nucleic acid from a sample solution, comprising:
a first container having an opening portion;
a second container having an opening portion; and
a solid phase for the adsorption of nucleic acid,
wherein a communication opening is formed via which the first container and the second container are communicated with each other,
wherein the solid phase is disposed within the communication opening.

13. The nucleic acid isolation instrument according to claim 12, comprising a disc-shaped rib formed at the opening portions of the first container and the second container.

14. An instrument for isolating nucleic acid from a sample solution, comprising:
a solid phase for the adsorption of nucleic acid;
a first container having a first opening portion, the first container further comprising an opening formed in its bottom in which the nucleic acid adsorption means is disposed; and
a second container having a second opening portion, wherein the second container is adapted to accommodate the first container.

15. The nucleic acid isolation instrument according to claim 14, comprising a disc-shaped rib formed at the opening portions of the first container and the second container.

16. An apparatus for isolating nucleic acid from a sample solution, comprising:
adsorption means for the adsorption of nucleic acid in the sample solution;
a nucleic acid isolation means comprising a first container portion and a second container portion that are communicated with each other via the adsorption means;
means for loading and unloading a solution into and out of the nucleic acid isolation instrument; and
pressurization/depressurization means for pressurizing the inside of one of the first container portion and the second container portion of the nucleic acid isolation means while depressurizing the inside of the other container portion.

17. The nucleic acid isolation apparatus according to claim 16, wherein the pressurization/depressurization means comprises a first syringe pump connected to the first container portion and a second syringe pump connected to the second container portion.

18. The nucleic acid isolation apparatus according to claim 16, wherein the loading/unloading means: loads the sample solution into the nucleic acid isolation means; unloads, after a pressurization/depressurization operation by the pressurization/depressurization means, the solution out of the nucleic acid isolation means; loads a washing solution into the nucleic acid isolation means; unloads, after a pressurization/depressurization operation by the pressurization/depressurization means, the washing solution out of the nucleic acid isolation means; loads an eluate into the nucleic acid isolation means; and unloads, after a pressurization/depressurization operation by the pressurization/depressurization means, the eluate out of the nucleic acid isolation means.
